(19) United States
(12) Patent Application Publication
(10) Pub. No.: US 2014/0058339 A1
(43) Pub. Date: Feb. 27, 2014

(54) METHOD AND APPARATUS FOR SELF-DOSING AND SELF-ADMINISTERING PHARMACEUTICAL COMPOSITIONS

(71) Applicants: Mark L. Baum, Solana Beach, CA (US); Joachim P.H. Schupp, San DIego, CA (US)

(72) Inventors: Mark L. Baum, Solana Beach, CA (US); Joachim P.H. Schupp, San DIego, CA (US)

(21) Appl. No.: 13/592,300

(22) Filed: Aug. 22, 2012

(51) Int. Cl. A61M 35/00

(52) U.S. Cl. 604/290; 604/310

(57) ABSTRACT

A method for self-dosing and self-administering a pharmaceutical or a cosmetic composition is provided. The method utilizes a three-dimensional dispensing device configured for receiving the composition. The dispensing device includes a bottom element having an inner surface and an outer surface and two side elements integrated with the bottom element, each side element being set at an angle to the bottom element.

### FIELD OF THE INVENTION

This invention relates generally to the field of medical devices. More specifically, the invention relates to a method and a device for self-dosing and self-administering of pharmaceutical or cosmetic creams, pastes, ointments, jellies or gels for transdermal applications.

### BACKGROUND

When pharmaceutical or cosmetic compositions are applied transdermally, there is frequently a need to have application devices which allow to accurately dose and deliver active drugs and inactive ingredients as a vehicle to the patient. Such need is particularly acute when dosing and delivery is conducted by the patient himself or herself, i.e., for self-dosing and self-administration of a topical cream, gel, emulsion, solution or other ointment carrier which ensures a unidirectional transfer of these to and/or through the skin.

Conventionally, ointments or creams are applied to a given area of the body such as the chest by squeezing a predetermined amount of ointment from a tube onto a piece of paper and applying the paper with the ointment loaded thereon to the body, the ointment providing the necessary adhesion of the paper to the body. Using applicators also allows to keep ointment or cream off the person's hands when applying.

Generally, typical paper applicators that are currently used consist of a ruled piece of waxed paper. When a person wishes to apply a given quantity of an active ingredient carried by the ointment or other solid or semi-solid formulation, one squeezes a given amount of the ointment, e.g., 1 g, from a tube onto the ruled piece of waxed paper in accordance with the drug manufacturer's package directions, followed by transferring the so squeezed ointment to the skin.

However, the formulation squeezed on a piece of paper represents a three-dimensional product, and the piece of paper used as the applicator is a two-dimensional device. This disparity frequently leads to errors in applying the correct volume of drug and quantity of active ingredient. There is much variability in squeezing a given amount of the ointment from a tube and the quantity varies with the force used to squeeze the tube and with an angle at which the composition is transferred from the tube to the paper. The quantity of the material put on the applicator also depends on other variables that are hard to even identify much less to quantify.

Accordingly, there is a need for improved methods and devices for self-dosing and self-administering of pharmaceutical or cosmetic creams, pastes, ointments, jellies or gels. To the best of the inventors' knowledge, such improved devices and methods have not been provided in the art. The present application provides some of such improved methods and devices.

### SUMMARY

According to one aspect of the invention, a method for self-dosing and self-administering a pharmaceutical or a cosmetic formulation is provided. Both human medical and veterinary uses are within the scope of the invention. The method utilizes a three-dimensional dispensing device configured for receiving the formulation. This dispensing device includes a bottom element having an inner surface and an outer surface; two side elements integrated with the bottom element, each side element being set at an angle to the bottom element, each side element also having an inner surface and an outer surface; and optionally two lip elements, each lip element being integrated with each of the two side elements.

Accordingly, in this aspect of the invention the bottom element and the two side elements define an inner space contained between them, and the bottom element and each of the two side elements comprise a central area for receiving the formulation. After dispensing the required volume of the formulation onto the central area of the device, the user then flattens and unfolds the device into a two-dimensional shape, by pressuring down the two side elements and optionally the lip element followed by transferring the formulation from the dispensing device to the area of the user's body that is in need of treatment.

According to another aspect of the invention, the formulation is selected from the pharmaceutically acceptable or cosmetically useful creams, pastes, ointments, jellies or gels, which typically have the dynamic viscosity of at least about 2,000 centipose.

According to yet another aspect of the invention, the dispensing device is fabricated of a flexible inert material, such as, e.g., pharmaceutically or cosmetically acceptable paper, plastic or a metal foil.

According to another aspect of the invention, a kit for self-dosing and self-administering a formulation comprising a pharmaceutical or a cosmetic composition is provided. The kit includes a three-dimensional dispensing device configured for receiving the formulation (the device further includes instructions for use and disposal imprinted on it) and a container containing the formulation.

Other aspects and specific features of the devices and methods of the invention are described in more detail below, in the Detailed Description portion of the instant application.

### BRIEF DESCRIPTION OF THE DRAWINGS

FIG. 1 depicts schematically an exemplary device and method, according to one embodiment of the present invention.

FIG. 2 is a cross-sectional view along the line A-A of the device shown by FIG. 1.

FIG. 3 is a cross-sectional view of an exemplary device, according to another embodiment of the present invention.

FIG. 4 is a cross-sectional view of an exemplary device, according to yet another embodiment of the present invention.

FIG. 5 is a cross-sectional view of an exemplary device, according to another embodiment of the present invention.

FIG. 6 depicts schematically the device shown on FIG. 1, when filled with the formulation that is used.

FIG. 7 depicts schematically a top view of the device shown on FIG. 1, when unfolded.

### DETAILED DESCRIPTION

It is to be understood that both the foregoing general description and the following detailed description are exemplary and explanatory only and are not restrictive of the invention claimed. As used herein, the use of the singular includes the plural unless specifically stated otherwise.

### Terms and Definitions

As used herein, “or” means to include both the meaning of “or” and the meaning of “and,” unless specifically stated otherwise. Furthermore, use of the term “including” as well as other forms, such as “includes” and “included,” is understood as “comprising” and “comprises” and is not limiting. In other words, the terms “including “includes” and “included” are considered being synonymous to the terms “comprising,” “comprises” and “comprised.” The section headings used herein are for organizational purposes only and are not to be construed as limiting the subject matter described.

The term “about” as used herein means that a number referred to as “about” comprises the recited number plus or minus 1-10% of that recited number. For example, “about” 1 gram can mean 0.95-1.05 gram or as few as 0.99-1.01 grams depending on the context.

Whenever it appears herein, a numerical range such as “1 to 20” refers to each integer or fractional unit thereof in the given range; for example and without limitation to a specific range content, “1 to 20 mm” means that a specified measurement can be 1 mm, 2 mm, 3 mm, etc., up to and including 20 mm, while 1.1 to 20.0 mm means that a specified measurement can be 1.1 mm, 1.2 mm, 1.3 mm, etc., up to and including 20.0 mm.

The term “pharmacologically active substance” refers to any chemical or biological compound or substance that affects the physiology or the function of a body of a human or an animal.

The term “pharmaceutically acceptable” refers to any carrier which does not interfere with effectiveness of the biological activity of the active ingredient and that is not toxic to the host to which it is administered.

The term “pharmaceutical composition” refers to a composition which may include one or several compounds or substances, for therapeutic use, whose application involves a chemical or physicochemical interaction with a physiological system of the host to which it is administered.

The term “cosmetics composition” refers to a composition which is to be applied to the human or animal body for cleansing, beautifying, promoting attractiveness, or altering the appearance without affecting the body's structure or functions. The term “cosmetics composition” does not include soap.

The term “cosmetically useful” refers to compositions which, when applied, are capable of cleansing, beautifying, moisturizing, promoting attractiveness, or altering the appearance of human or animal body without affecting the body's structure or functions.

The term “cosmetically acceptable” refers to a material that does not interfere with effectiveness of a cosmetics composition.

The term “formulation” refers to a medical or veterinarian preparation fabricated according to a specific formula.

The term “user” refers both to a human who uses the methods and devices of this invention and an animal to which a human administers a formulation.

The term “dynamic viscosity” refers to a measure of the resistance to flow of a fluid under an applied force.

The term “paper” refers to thin sheets (typical thickness being less than about 2 mm) of a substance made from wood pulp, rags, straw or other fibrous material, used to bear writing or printing inscribed or printed thereupon.

The term “plastic” refers to a material comprising synthetic or semi-synthetic organic amorphous solids, typically, polymers of high or medium molecular mass (between about 10,000 Daltons and 1,000,000 Daltons or higher).

The term “metal” refers to a solid material that is typically hard, shiny, malleable, fusible, ductile and electrically and thermally conductive.

The term “transparent” refers to a quality of a material that allows it to transmit light so that objects or images can be seen as if there were no intervening material.

The terms “crimp” and “crimping” refer to the process of pressing or pinching an object to form small regular folds or ridges.

The term “inert material” refers to a material that is devoid of active properties, is not chemically, physicochemically or biologically reactive and is unable or unlikely to change in any way the properties of another material with which it comes into a contact.

The term “hypoallergenic” refers to a material that is designed to eliminate, reduce or minimize the possibility of an allergic response when a human or animal body comes into a contact with it.

### Embodiments of the Present Invention

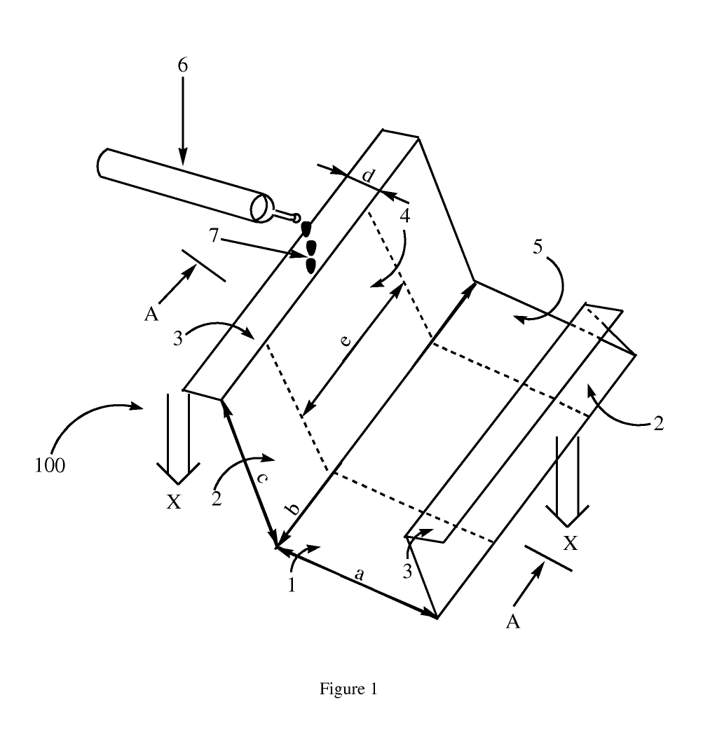

According to embodiments of the present invention, various devices and methods for self-dosing and self-administering of topical transdermal pharmaceutical or cosmetic creams, pastes, ointments, jellies or gels are provided. The devices of the present invention may be generally described with the reference to FIGS. 1-7 showing, but not limited to, certain exemplary embodiments of the invention.

More specifically, FIG. 1 depicts schematically a three-dimensional dispensing device 100 that is configured for receiving a formulation 7 comprising a pharmaceutical or a cosmetic composition. Device 100 shown on FIG. 1 can be made by converting a flat, thin two-dimensional sheet of a flexible inert material into a three-dimensional shape by folding and crimping the sheet to form device 100 having the parts discussed below. Non-limiting examples of suitable flexible inert materials that can be used include pharmaceutically or cosmetically acceptable paper, plastic or a metal foil.

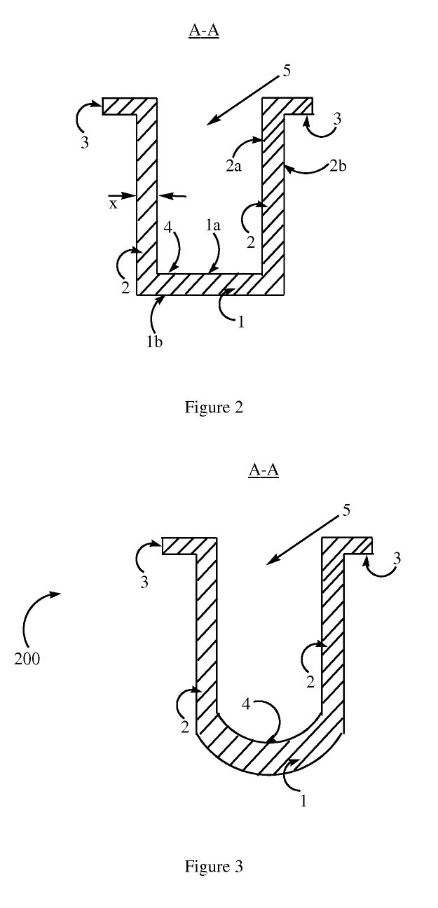

Those having ordinary skill in the art will select the specific material of which device 100 can be fabricated as well as choose the thickness of the thin sheet of a flexible inert material selected for fabricating device 100. When choosing the thickness, those having ordinary skill in the art will keep in mind that in many embodiments the device is to remain transparent as discussed in more detail below, as well as crimpable and reversibly foldable and unfoldable. In some exemplary, non-limiting embodiments the thickness (shown as “x” on FIG. 2) is less than about 2 mm, for example, between about 100 μm and about 2,000 μm, typically between about 200 μm and about 250 μm (for paper).

Device 100 so fabricated out of a thin sheet of a flexible inert material includes a bottom element 1 having an inner surface 1a and an outer surface 1b (FIG. 2). Integrated with bottom element 1 are two side elements 2. Each side element 2 is positioned at an angle to bottom element 1, and each side element 2 also has an inner surface 2a and an outer surface 2b (FIG. 2).

Inner surfaces 1a and 2a can be optionally additionally treated, e.g., by having a thin layer of wax deposited on them, to ensure that inner surfaces 1a and 2a remain inert and to prevent all possible interactions of these surfaces with formulation 7 when the latter is deposited on inner surfaces 1a and 2a, as discussed below.

Those having ordinary skill in the art will select the specific length (shown as “b” on FIG. 1) and width (shown as “a” on FIG. 1) of bottom element 1 and the height of the side elements 2 (shown as “c” on FIG. 1), thus determining the length, width and height, respectively, of the entire device 100.

In some exemplary, non-limiting embodiments, the length “b” of the dispensing device 100 is, independently of other dimensions, between about 70 mm and about 100 mm, such as between about 75 mm and about 85 mm, for example, about 80 mm. In some exemplary, non-limiting embodiments, the width “a” of the dispensing device 100 is, independently of other dimensions, between about 1 mm and about 50 mm such as between about 5 mm and about 30 mm, for example, about 8 mm. In some exemplary, non-limiting embodiments, the height “c” of the dispensing device 100 is, independently of other dimensions, between about 5 mm and about 35 mm such as between about 10 mm and about 20 mm, for example, about 25 mm.

Furthermore, for the convenience of the user, device 100 may optionally include two lip elements 3 (shown on FIGS. 1-7) where each lip element 3 is integrated with the corresponding side element 2. Either or both of the lip elements 3 may be utilized by the user for holding device 100 when dispensing formulation 7, as explained below The width of each lip element 3 (shown as “d” on FIG. 1) can be the same or different. Those having ordinary skill in the art can select the most appropriate width “d.” In one exemplary, non-limiting embodiment the width “d” may be between about 5 mm and about 50 mm such as between about 10 mm and about 30 mm, for example, about 25 mm.

Bottom element 1 and the two side elements 2, therefore, define an inner space 5 contained between them, as can be seen on FIGS. 1-6. This inner space 5 can have a variety of geometric forms and shapes. For example, in one exemplary, non-limiting embodiment each side element 2 can be positioned at the right angle to bottom element 1, accordingly forming inner space 5 that is being shaped as a rectangular parallelepiped, as shown on FIG. 2.

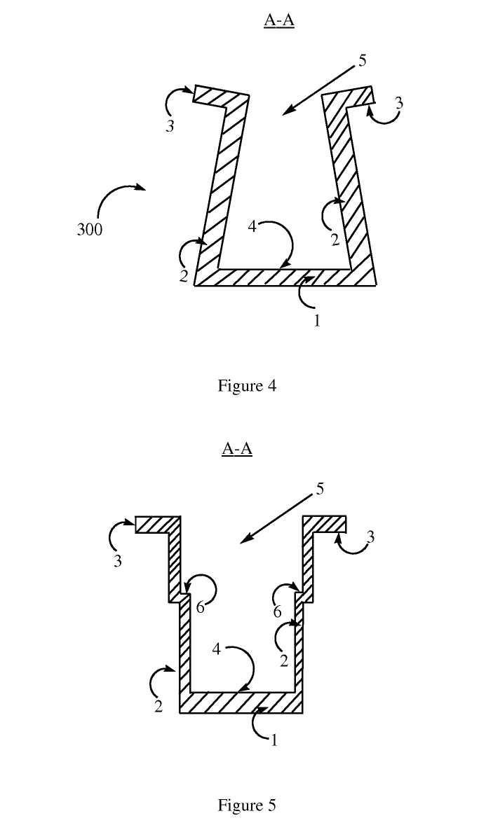

In another exemplary, non-limiting embodiment each side element 2 can be also positioned at the right angle to bottom element 1, but bottom element 1 can be semi-circular in the cross-section instead of flat discussed above. Accordingly, inner space 5 can be shaped as a semi-cylinder, as shown by FIG. 3. In yet another exemplary, non-limiting embodiment instead of being positioned at the right angle to bottom element 1, each side element 2 can be also positioned at an acute angle to bottom element 1. Accordingly, inner space 5 can be shaped as a truncated prism, as shown by FIG. 4.

Other types of figures can characterize the shape of inner space 5. In general, therefore, non-limiting examples of shapes that inner space 5 of the dispensing device 100 may conform to include a rectangular prism, a rectangular parallelepiped, a cuboid, a semi-cylinder, a truncated cone, a truncated prism or a truncated pyramid.

In yet additional embodiments, the dispensing device 100 may be fabricated in such a way that that the width of inner space 5 is not uniform along the device's height dimension “c.” In other words, one or both of side elements 2 can comprise one or more notches. In one exemplary, non-limiting embodiment shown by FIG. 5, both side elements 2 include one notch 6. Having one or more notches allows the use to vary doses of formulation 7 when formulation 7 is dispensed to device 100, as explained below. Those having ordinary skill in the art can select the number of notches as well as the depth of each notch 6, if such embodiments are to be employed.

As can be seen from FIGS. 1, 6 and 7, bottom element 1 and each of the two side elements 2 comprise a central area 4 for receiving formulation 7. Those having ordinary skill in the art can determine what the desired width “e” of central area 4 is. In some exemplary, non-limiting embodiments, the width “e” may be between about 5 mm and about 100 mm, such as between about 25 mm and about 35 mm, for example, about 30 mm. In other embodiments, the width “e” may be such as to match the dimension of the orifice of the tube 6. Since the dimension of the orifice of the tube 6 can be between about 1 mm and about 8 mm as described below, in these embodiments the width “e” may be also between about 1 mm and about 8 mm.

The width “e” of central area 4, the width “a” and the height “c” of device 100 together determine the volume of formulation 7 that is being dispensed. Since the density of the composition is fixed and depends solely on the properties of the composition itself, the weight of formulation 7 that is being dispensed is thus predetermined and precise.

To improve the visibility and to make central area 4 more discernible, it can be colored using a bright color ink such as red ink as a visual aid. This requires the material of which device 100 is made to be transparent at the thickness that is utilized. The ink is typically applied to outer surfaces 1b and 2b at the bottom and the sides of device 100, over central area 4. The brightness and the color of the ink should be selected by those having ordinary skill in the art in such a way that the colored portion can be seen through the material of which device 100 is fabricated.

In other words, when the ink is applied to outer surfaces 1b and 2b, a user of device 100 should be able to see the colored area when looking at device 100 from the direction of inner surfaces 1a and 2a. The instructions for use and disposal can be printed as a mirror image, so as to be easily readable when a user is looking at the device 100 from the direction of the inner surfaces 1a and 2a. Samples of the text carrying the instructions can be typically, but is not limited to, the font Arial, bold, with font sizes 8-18. The instructions can carry the following messages: “Please see Dosing and Disposal Instructions for Details on Use,” or “Please Fill the Entire Indicated Area With Medication.” Other message(s) can be alternatively imprinted if desired.

If desired, alternatively, both the ink and the instructions can be applied to inner surfaces 1a and 2a of the device 100, in which case the ink used for the coloring of central area 4 and the ink used for printing the instructions should be inert and hypoallergenic.

In some embodiments, to make the central trough of specific, predetermined dimensions an algorithm can be used that will automatically fold the material to fabricate the device 100, to allow the correct amount of the composition to be dispensed given the required weight of the material for a dose of the same.

Device 100 may constitute a part of a kit for self-dosing and self-administering formulation 7. The kit includes a three-dimensional dispensing device 100 configured for receiving the formulation, as described above, and container 6 (FIG. 1) containing the formulation. One non-limiting example of container 6 that may be used is a squeezable tube. Other kinds of containers can be used instead if desired.

The kit further includes instructions for use imprinted on device 100. As stated above, the instructions can be imprinted either on outer surfaces 1b and 2b or on the inner surfaces 1a and 2a. In some embodiments of the kit, the dispensing device 100 is disposable (i.e., is intended only for a single use). In other embodiments of the kit, the dispensing device 100 is reusable (i.e., is suitable for multiple uses).

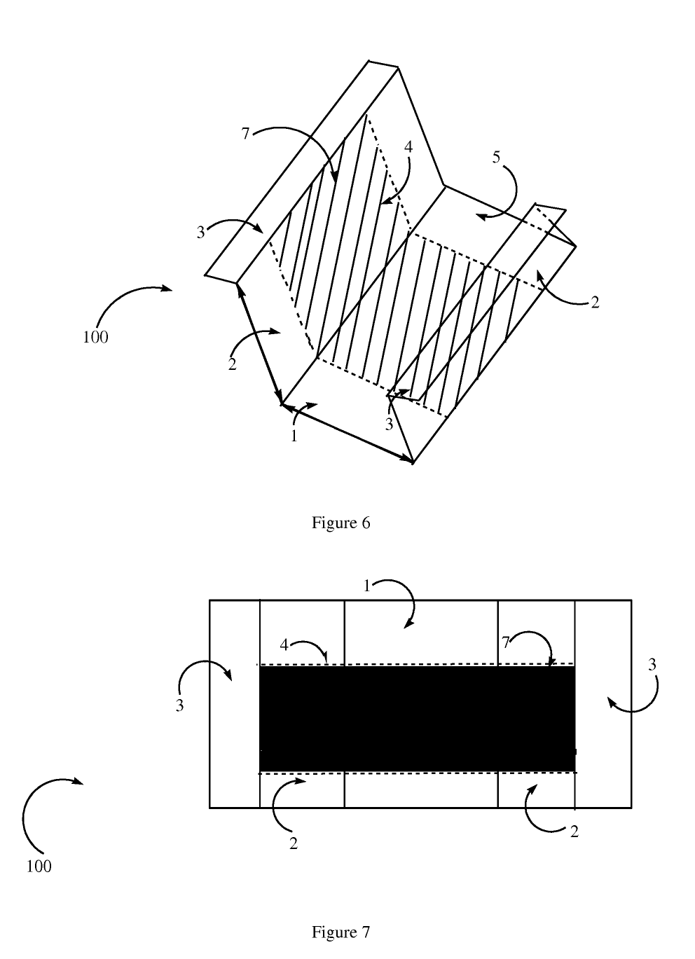

Having described the dispensing device 100 and a kit of which the device 100 is a part, we now turn to the description of methods of using the device and the kit. The user dispenses formulation 7 from container 6 (e.g., by squeezing the squeezable tube) and directs formulation 7 onto central area 4 of device 100, until the entire volume of the portion of internal space 5 that is limited by central area 4 and the height “c” is filled with formulation 7. This process is schematically illustrated on FIG. 1, and the view of the device filed with formulation 7 (shown as a cross-hatched area) is provided on FIG. 6. Whether this volume is completely or only partially filled with formulation 7 can be checked visually. This check is facilitated if central area 4 is colored as described above.

As mentioned above, the density of formulation 7 is constant for a given formulation, and the above-described process of dispensing formulation 7 leads to a predetermined volume being dispensed in the above-described fashion. Therefore, the user can dispense a predetermined and precise quantity of formulation 7. Any required amount of any formulation amount may be so dispensed, e.g., between about 100 mg and about 5 g.

In some embodiments, varying doses of formulation 7 may be dispensed, if desired. For example, the device illustrated by FIG. 5 includes notch 6. As can be seen, internal space 5 comprises two distinct areas, a smaller area below the notch and a larger area above it. This arrangement allows dispensing a smaller dose of formulation 7 by filling only the area up to the notch, or a larger dose, by filling internal space 5 up to lip 3. As explained above, more than one notch can be formed in one or both of the side elements 2 thus allowing device 100 to be used for self-dosing and administering multiple doses.

Those skilled in the art will understand that in order to allow device 100 to retain the dispensed formulation 7, the formulation should be solid or semi-solid. In some embodiments, the formulation can have the dynamic viscosity of at least about 2,000 centipoise, such as between about 5,000 centipoise and about 500,000 centipoise, e.g., between about 66,000 centipoise and about 300,000 centipoise, for example, between about 140,000 centipoise and about 232,000 centipoise.

A variety of such formulations may be used, in the form of pharmaceutically or cosmetically acceptable creams, pastes, ointments, jellies or gels, each containing a pharmaceutically effective quantity of a pharmacologically active substance or cosmetically useful substance, with no limitations on the type and king of such pharmacologically active or cosmetically useful substances. Indeed, the devices and the methods described above are suitable for the administration of any drug which when applied to an area of intact skin acceptable to physician and patient can elicit an adequate systemic therapeutic effect. The devices and the methods described above, therefore, have general applicability for achieving a constant therapeutic effect with any drug or therapeutic agent that is permeable to the skin and which experiences a high degree of liver metabolism.

Some exemplary, non-limiting examples of suitable pharmacologically active substances include ketoprofen, cyclobenzaprine, gabapentin, diclofenacnitroglycerol, clonidine, prochlorperazine, scopolamine and hormones such as testosterone or secretin. One example of a suitable formulation that can be used with the three-dimensional dispensing device of the present application is 10% ketoprofen in pluronic lecithin organogel.

Those having ordinary skill in the art can also adapt the devices and the methods described above for application of any products onto mucosal surfaces of cavities such as intra-orally, intra-nasally, intra-auricular, vulvo-vaginal, intra-urethrally or rectally.

As shown on FIG. 1, after dispensing the required volume of formulation 7 onto central area 4 of device 100, the user then flattens and unfolds the device into a two-dimensional shape (shown schematically on FIG. 7), by pressuring two side elements 2 and optionally lip elements 3 in direction “X.” Formulation 7 can then be transferred from the dispensing device to the area of the user's body that is in need of treatment.

Although the foregoing invention has been described in some detail by way of illustration and example for purposes of clarity and understanding, it will be apparent to those of ordinary skill in the art in light of the teaching of this invention that certain changes and modifications may be made thereto without departing from the spirit or scope of the appended claims.

What is claimed is:

1. A method for self-dosing and self-administering a formulation comprising a pharmaceutical or a cosmetic composition, the method comprising:
(a) providing a three-dimensional dispensing device configured for receiving the formulation, the dispensing device comprising:
(1) a bottom element having an inner surface and an outer surface;
(2) two side elements integrated with the bottom element, each side element being set at an angle to the bottom element, each side element having an inner surface and an outer surface; and
(3) optionally two lip elements, each lip element being integrated with each of the two side elements,
wherein the bottom element and the two side elements define an inner space contained therebetween, with the further provisos that:
the bottom element together with the two side elements comprise a central area for receiving the formulation,
the dispensing device is capable of being flattened by pressuring the bottom element, the two side elements and optionally the lip element into a two-dimensional shape;
(b) dispensing the required amount of the formulation onto the central area of the dispensing device;
(c) unfolding the dispensing device into the two-dimensional shape; and
(d) transferring the formulation from the dispensing device to the area of the user's body that is in need of treatment,
to self-dose and self-administer the formulation thereby.

2. The method of claim 1, wherein the formulation is selected from the group consisting of pharmaceutically acceptable or cosmetically useful creams, pastes, ointments, jellies and gels.

3. The method of claim 1, wherein the formulation comprises a pharmaceutically effective quantity of a pharmacologically active substance.

4. The method of claim 3, wherein the pharmacologically active substance is selected from the group consisting of ketoprofen, cyclopenzaprine, gabapentin, diclofenac, nitroglycerol, clonidine, prochlorperazine, scopolamine and hormones such as testosterone or secretin.

5. The method of claim 2, wherein the formulation has the viscosity of at least about 2,000 centipoise.

6. The method of claim 5, wherein the formulation has the viscosity between about 5,000 centipoise and about 500,000 centipoise.

7. The method of claim 1, wherein the dispensing device is fabricated of a flexible inert material.

8. The method of claim 7, wherein the flexible inert material is pharmaceutically or cosmetically acceptable and is selected from the group consisting of paper, plastic and a metal foil.

9. The method of claim 1, wherein the thickness of the dispensing device is less than about 2 mm.

10. The method of claim 1, wherein the length of the dispensing device is between about 70 mm and about 100 mm.

11. The method of claim 1, wherein the width of the dispensing device is between about 1 mm and about 50 mm.

12. The method of claim 1, wherein the height of the dispensing device is between about 5 mm and about 35 mm.

13. The method of claim 1, wherein the width of the central area of the dispensing device is between about 5 mm and about 100 mm.

14. The method of claim 1, wherein the amount of the composition dispensed onto the central area of the dispensing device is between about 100 mg and about 5 g.

15. The method of claim 1, wherein the inner space of the dispensing device conforms to a shape selected from the group consisting of a rectangular prism, a rectangular parallelepiped, a cuboid, a semi-cylinder, a truncated cone, a truncated prism and a truncated pyramid.

16. The method of claim 1, wherein each of the two side elements comprise at least one notch so that the width of the inner space is not uniform along the inner space's height dimension.

17. The method of claim 1, wherein the outer surface of at least one of the bottom element and two side elements includes instructions printed thereupon in a minor image manner.

18. A kit for self-dosing and self-administering a formulation comprising a pharmaceutical or a cosmetic composition, the kit comprising:
(a) the three-dimensional dispensing device of claim 1; and
(b) a container containing the formulation,
wherein the dispensing device further includes instructions for use imprinted thereupon.

19. The kit of claim 18, wherein the dispensing device is disposable.

20. The kit of claim 18, wherein the dispensing device is reusable.
